(19) United States
(12) Patent Application Publication
(10) Pub. No.: US 2021/0161531 A1
(43) Pub. Date: Jun. 3, 2021

(54) SURGICAL STAPLER

(71) Applicants: Joaquin Sabaris Vilas, Barcelona (ES); Manuel Trias Folch, Barcelona (ES)

(72) Inventors: Joaquin Sabaris Vilas, Barcelona (ES); Manuel Trias Folch, Barcelona (ES)

(21) Appl. No.: 17/048,681

(22) PCT Filed: Apr. 17, 2019
(86) PCT No.: PCT/ES2019/070269

§ 371 Date: Oct. 19, 2020

(30) Foreign Application Priority Data
Apr. 18, 2018 (ES) U201830556

(51) Int. Cl. A61B 17/115; A61B 17/072; A61B 5/026

(52) U.S. Cl. CPC A61B 17/115; A61B 2017/07285; A61B 5/0261; A61B 17/072

(57) ABSTRACT

The stapler of the invention is a surgical stapler/cutter that allows latero-lateral intestinal anastomosis, for both open surgery and laparoscopic surgery, at a distance from the openings used to insert the instrument. The stapler is based on the conventional structure of this type of stapler, and one of the fundamental features thereof is that an interspace (12) that determines a space for inserting the free ends (13, 13′) of the tissues (8, 8′) to be joined, is determined between an articulation shaft (3) of the two jaws (4, 5) of the stapler and the front end at which are disposed forms (6) and vertically movable teeth (9) on which staples (7) are disposed. This prevents the staples for the anastomosis and those for the removal of the pathological area from crossing when the area to be removed is cut. The invention also provides for the inclusion, in the described inter-space (12), of an Infrared Data Association (IrDA) device for testing blood flow that transmits a signal to a luminous signalling device which is visible to the user and which allows the degree of tissue vascularisation to be assessed.

### OBJECT OF INVENTION

The present invention refers to a surgical stapler, specially designed to carry out operations of mechanical latero-lateral intestinal anastomosis.

The purpose of the invention is to provide a stapler with a mechanical functioning such that it allows to perform complete anastomosis at a certain distance from the points of introduction of it to the intestinal light, so that it stops a small stretch of intestine well irrigated, between the anastomosis and the section area for the removal of the surgical piece, avoiding crossover between the staples of latero-lateral anastomosis and that of the section of the surgical part being removed.

It is also the object of the invention that the stapler has self-adjusting internal cutting media, in accordance with European operator safety regulations, which allows longitudinal cutting of intermediate tissues within the stapling, thus facilitating the exact area of passage to the object of anastomosis.

### BACKGROUND OF INVENTION

Mechanical latero-lateral intestinal anastomosis is a technique that is very commonly used in general and digestive surgery, both in open and laparoscopic surgery (esophagus, stomach, obesity, colon, etc. . . . ).

When mechanical sutures are used, intra-operative contamination is lower than when performed with manual techniques with suture thread, since the exposure of the open intestine and the time of the technique is lower, making it one of the most used options.

In this technique, latero-lateral anastomosis is usually done with a stapler-cutter instrument through small incisions in the distal intestinal handles, where the instrument that joins the two intestinal walls with various parallel staples is introduced, and makes an incision between them, which will form the light of anastomosis.

The corresponding intestinal section, which is usually performed including the holes used for the introduction of the mechanical instrument into the exeresis part, must then be sectioned and removed.

Anastomotic leakage is not uncommon and can reach 8% with immediate morbidity (septis, peritonitis, fistulas, reoperations, etc. . . . ), and late (incisional hernias, enclosure of fistulas and stomata, etc.), whose mortality can reach 3%.

One of the causes of anastomotic leakage is the possible ischemia of the intestinal wall of the area of anastomosis, either because the mesos that have been sectioned to remove the pathological area exceed the limits of the good or poor irrigation of the intestinal walls and are difficult to evaluate, or because the crossing of the staples of the anastomosis and those of the section line of the piece to be removed produce ischemia Local. Some surgeons add individual stitches or continuous sutures to reperitonize, cover, and protect anastomosis, even if there are doubts about whether this increases local ischemia or produces holes that can develop into perforations. (Today, intraoperative green-indocyanine injection is even used to confirm good local irrigation.)

When making latero-lateral anastomosis with mechanical sutures through small holes, then these holes must be closed, in some cases through the use of manual sutures or with mechanical sutures, or include them in the resection piece, which requires the section of tissues including part of anastomosis. In this case, a cross-crossing of mechanical sutures that could generate important local ischemia could be influenced.

This crossover or overlapping of staples is due to the mechanics of use of current surgical ones.

Specifically, these staplers start from a standard scissor format, in which both assemblable arms on the shaft, allow stapling functionality; one of them, being the support for the placement of the staple load, with the internal cutting blade protected and the other, acting as a closing jaw for the deformation of the staples that sew both fabrics, so that the closing action of the scissors under pressure, together with the axial displacement of an internal thrust cam (drive), create the mechanical suture by deformation of the staples and the internal cutting of the layers of healthy tissue, attached to the object of anastomosis, valid for the passage of liquids and solids through the new junction point of both healthy intestines.

The problem with this type of stapler is because the arrangement and start of the stapling of its jaws causes the start of the stapling and cutting line to be made from the holes themselves for the introduction of the mechanical instrument into the handles, so that, when the instrument is removed, the anastomosis is in line with those holes. This forces the closure of them, either in continuity with anastomosis or the exeresis of them next to the piece to be removed, including part of the anastomosis itself.

### DESCRIPTION OF THE INVENTION

The surgical stapler that is recommended solves in a fully satisfactory way the problem previously exposed, both for staplers/cutters in open and laparoscopic surgery, based on a simple but very effective solution that allows anastomosis to be performed at a certain distance from the points of introduction of the instrument to the intestinal light, so that the staples form a complete oval allowing when cutting between the two intestinal handles to anastomosar, these fall within the internal limits of these staples, forming an anastomosis in the form of a full sealing oval, and, on the other hand, thanks to the lower height or rather, the widening inside the jaws area of the stapler between the stretch that performs the anastomosis and the grip of the stapler avoid compressing the intestine in this section.

To do this, and more specifically, the stapler of the invention, part of the conventional form described above, in which a device is manipulated as a stapler and continuous cutter from the shaft to the closure, another that initiates this action at a certain distance from the axis, leaving an internal and second space of irrigation of the joined fabric, at a sufficient safety distance between the entry point and the intended junction point, under the guarantees against ischemias and the prevention of drag or crossing of staples at the closure.

The fundamental differences are:

A) Performing complete anastomosis;
B) Anastomosis is some distance from the insert holes of the mechanical instrument;
C) That, between the base of the stapler and anastomosis, the jaws leave a free space to avoid injury to the intestine included in this area, which will later be the section area of the tissue to be removed, and that, therefore, makes it unnecessary to have to include part of the anastomosis, avoiding the crossing of the two stapler lines, as was the case with the previous stapler models.
D) And finally it has an IrDA-type blood flow-check device, in the chamber (12), with an emitter (17) and a receiver (18), connected to a light signalling device (19) located on an external arm (1) in the user's sight.

To achieve these objectives, these technical contributions have been made:

1) The design of the staples and the corresponding cavities to form them, have a distribution that allows complete anastomosis;
2) According to the other of the characteristics of the invention, means of cutting the tissue have been provided to perform anastomosis, which for safety reasons are only visible and act, at the time of stapling manoeuvre, and consisting of the inclusion in the internal guides of the stapler load, on one of the stapling arms, of a tilting blade, which, in an inoperative situation, will remain hidden in that jaw and which is intended to be operated in a synchronized manner with the grape closing manoeuvre, that is, that the blade becomes tilting and consequently emerges, by means of the thrust bar itself in cam functions for the grinding wheel against the staples, only during cutting among the rows of staples.

Thus, when the stapling is actuated by means of the thrust trigger, the inner cam that moves along the consumable load, acts on a lateral teton of the tilting system of the cutting blade, causing it to rise and complete the longitudinal cut, from start to finish, while the anastomical ovoid is stapled.

Once the loading path is complete, the blade retracts so that it is hidden from the operator.

In this way, the cutting media of the stapler are safer, respect the safety regulations of the operator, in addition to carrying out a precise cut, exclusively between the stapled area, which allows to communicate both intestinal sections with the guarantees that conform to anastomosis, not affecting the initial un stapled section, on which the section of the tissue to be removed will subsequently be made, including the holes to insert the stapler.

3) The jaws of the stapler are narrower in the area that goes from its joint to the area of the staples, forming an inner bridge so that when the stapler is closed for the realization of anastomosis they do not in lesion the intestine between the holes and anastomosis.
4) An IrDA-type blood flow-check device is available in the chamber, which is defined near the articulation area of the two arms, with an emitter and receiver, connected to a light signalling device located on an external arm in the user's sight.

According to a variant of practical realization of the invention, it has been envisaged that the stapler of the invention can be adapted for use in laparoscopic surgeries, for which, starting from the same structuring, described above, it has been envisaged that the jaw corresponding to that which was the assisted arm is subsequently prolonged in a long arm in whose bosom is axially displacable the thrust bar that is finished in the aforementioned cam in the form of a sloping plane and that causes the sequential stapling of the fabrics, so that the bar is finished at its other end in a trigger thrust mechanism, implanted on the corresponding handle, which allows the manual and controlled operation of said mechanism.

For its part, the upper or pressing jaw, which shall be articulated with respect to the lower jaw, presenting the same configuration as regards its inner face, both in the means of guiding the staples, and in the chamber that is defined between the two jaws, and the means of checking blood flow and signaling, so that to operate that jaw it is intended to be required towards its opening position by effect of a spring associated with its tilting shaft, so that the upper jaw includes in correspondence with its anterior and upper limb, a cavity that is facing the upper edge of the aforementioned laparoscopic arm, so that, based on that upper area of the arm, it is axially and manually displacable a rod, at all times parallel to the laparoscopic arm and that, by virtue of this forced parallelism, makes it necessary that when it enters the cavity of the upper jaw, it is in store to swing that jaw in the lower direction, until it adopts a disposition entirely parallel to that arm, and that they correspond to the closing position on the lower jaw.

### DESCRIPTION OF DRAWINGS

To complement the description to be made below and in order to help a better understanding of the characteristics of the invention, in accordance with a preferred example of practical realization of the invention, is accompanied as an integral part of that description, a set of plans in which the following has been represented for illustrative and non-limiting purposes:

FIG. 1 Shows a side elevation and section view of a surgical stapler performed according to the object of the invention, in an initial phase of approximation to the tissues to be joined, in which it can be observed as in it is left a space or camera before which the tip of the tissues access, in order that the limb is not affected by the staples or jaws, with a view to subsequently making a stapling line on that area in another direction without overlapping staples.

FIG. 2.—Shows a view similar to that of the figure above, but in which the jaws of the stapler appear properly closed.

FIG. 3.—Displays a view similar to that in FIG. 2, but in which the push bar in cam functions of the stapler appears in an intermediate working position.

FIG. 4.—Shows a detail of the bond between the fabrics after both stapling lines have been made in different directions, and it can be observed that at the free end of the fabrics to be joined do not overlap the staples of one alignment with those of another.

FIGS. 5 and 6.—Show two expanded details of FIGS. 2 and 3 at the level of the stapler's jaws, so that they can more clearly observe their internal structure.

FIG. 7.—Shows a sequence of the anastomosis process with the traditional system

FIG. 8.—Shows a sequence of the anastomosis process with the process of the invention.

FIGS. 9 and 10.—Show views similar to FIGS. 1 and 2 but corresponding to a variant of realization of the invention in which the stapler includes means of remote operation, for example, for laparoscopic surgeries.

FIGS. 11 and 12.—Finally, show two expanded details of FIGS. 9 and 10 at the level of the drive media of the upper jaw of the stapler.

### PREFERENTIAL REALIZATION OF THE INVENTION

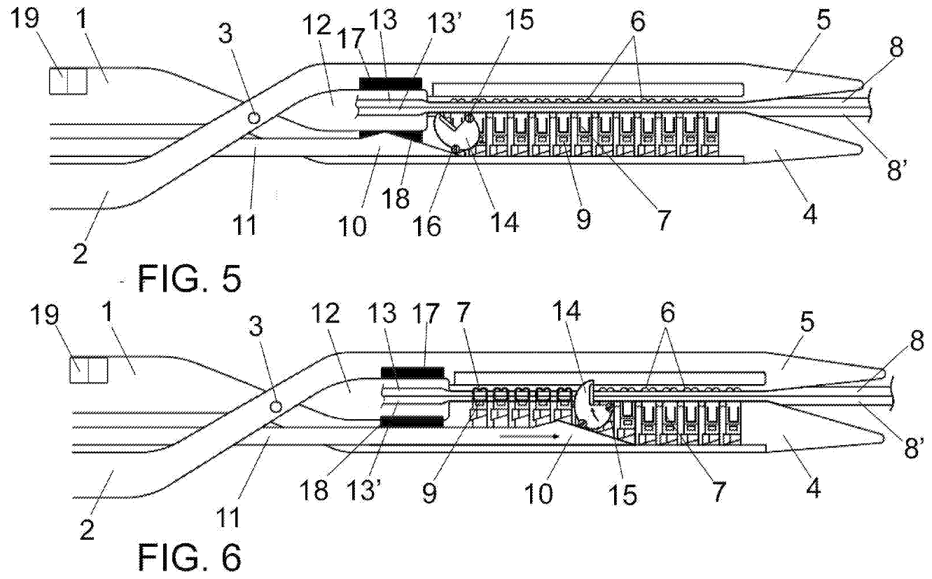

In view of the figures outlined, it can be observed how the stapler of the invention is constituted, as is conventional, applicable to open or laparoscopic surgeries, being of the type of those that are constituted from a device as scissors, or tilting closure in the case of laparoscopy, in which two arms (1-2) of roasting and pressing, articulated on one axis (3) and topped in jaw paths (4-5), so that in one of these jaws (5) a series of conformations (6) are established internally intended to plastically deform the staples (7) that are arranged on the jaw (4) causing them to curve inward once the tissues are crossed (8-8′) to be joined, for which, staples (7), initially set in “U”, are available on vertically scrollable grinding teeth (9) by the effect of a cam (10) in the form of a sloping plane in which a push bar (11) horizontally scrollable in the sine of the jaw (4) is finished, so that the displacement of the push bar (11) causes the sequential stapling of the tissues (8-8′), as shown in FIGS. 5 and 6.

Well, from this structuring, the stapler of the invention focuses its characteristics on the fact that between the axis of articulation (3) of the two jaws (4-5) and the initial end at which the aforementioned conformations (6) and grinding wheel are available vertically scrollable (9) on which staples are available (7), a camera (12) is defined as determinant of a space wide enough to allow the free ends (13-13′) of the fabrics to be joined (8-8′), a magnitude and a length greater than the width of the staples (7), in order to define a space between the free end of the staples and the start of the stapling line sufficient to allow to carry out with the same utensil a second line of stapling, perpendicular to it or where appropriate in another direction, without the first staple (7′) applied in this second stapling line (see FIG. 4) overlapping with the first of the staples (7) implanted in the first manoeuvre.

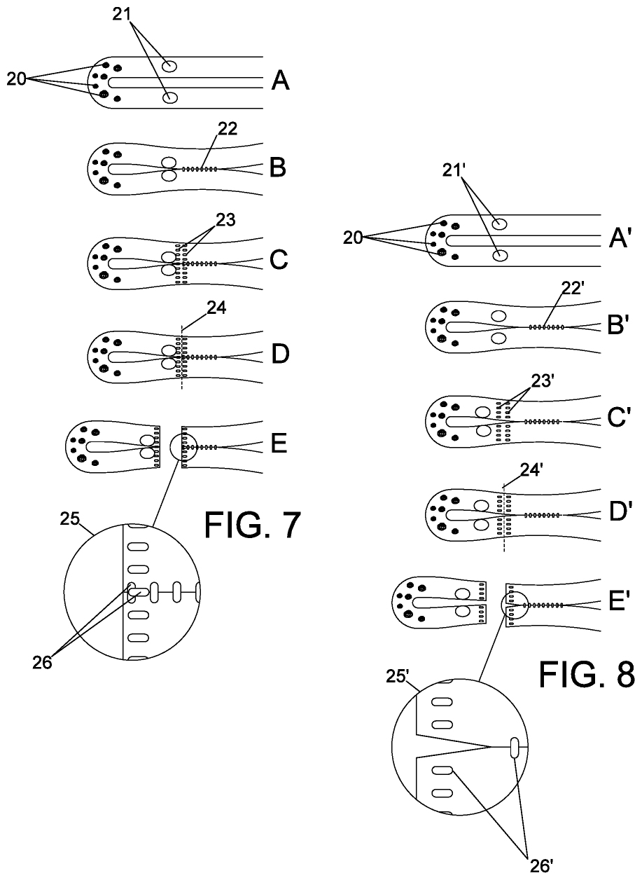

In FIG. 7 you can see the sequences of the pre-invention process, which are:

A) the part of the intestine is shown to anastomar, with the tumors (20) to be removed, and the holes (21) made to introduce the surgical stapler.
B) The first stapling (22) performed is displayed.
C) The second cross stapling is shown (23)
D) Cutting line shown (24)
E) The segment already sectioned with an extended detail (25) showing the overlap of the staples (26) is displayed.

FIG. 8 shows the sequences of the process proposed by the invention, which is characterized by having a sufficient space so that the cross stapling does not overlap the first stapling:

A) the part of the intestine is shown to anastomar, with the tumors (20′) to be removed, and the holes (21′) made to introduce the surgical stapler.
B) The first stapling (22′) performed is displayed.
C) The second cross stapling (23′)
D) Cutting line is displayed (24′)
E) The segment already sectioned with an extended detail (25′) is shown showing the non-overlapping of staples (26′).

As previously said, this prevents the crossing of staples in the sutures, thus avoiding possible anastomotic leaks.

As for the cutting media provided for in the stapler, these consist of a tilting blade (14), which, in an inoperative situation, will remain hidden the jaw (4), tilting with respect to a turning axis (15), which is axially scrollable in a longitudinal groove of the jaw, not represented in the figures, against the tension of a spring, counting this tilting blade (14) with a brass (16) intended to come into contact with the ramp or cam (10) of the thrust arm (11) during its axial displacement in the sinus of the jaw (4), which causes its initial tilting until it emerges in a cutting arrangement, position in which it will remain angularly unchanged, moving in conjunction with the cam (10) along the longitudinal groove in which its axis plays (15), thus obtaining safer means of cutting.

The surgical stapler, to ensure proper tissue irrigation, has in the chamber (12) an IrDA-type blood flow-checking device, with emitters (17) and infrared receptors (18) of infrared rays, of appropriate frequency, so that once faced by passing the rays the tissue (13-13′), sends the data to a signaller (19) for example green/red, located in the part of the stapler that is best visualized by the surgeon, in this way the correct position for subsequent sewn is ensured, so it could represent a useful solution that prevents local ischemias in this type of anastomosis, which can be one of the most important causes of failure of intervention by anastomotic leaks.

This new distal point rapper-cutter device is also applicable to latero-side anastomosis as the only bypass technique, which, represents an advantage, since anastomosis is performed remotely from the holes for the introduction of the mechanical device, and allows the closure of them without continuity with respect to anastomosis.

Thus, the object of the present invention is to avoid the lack of irrigation of anastomosis, especially by crossing the staples between the latero-lateral anastomosis and that of the section of the surgical piece that is removed.

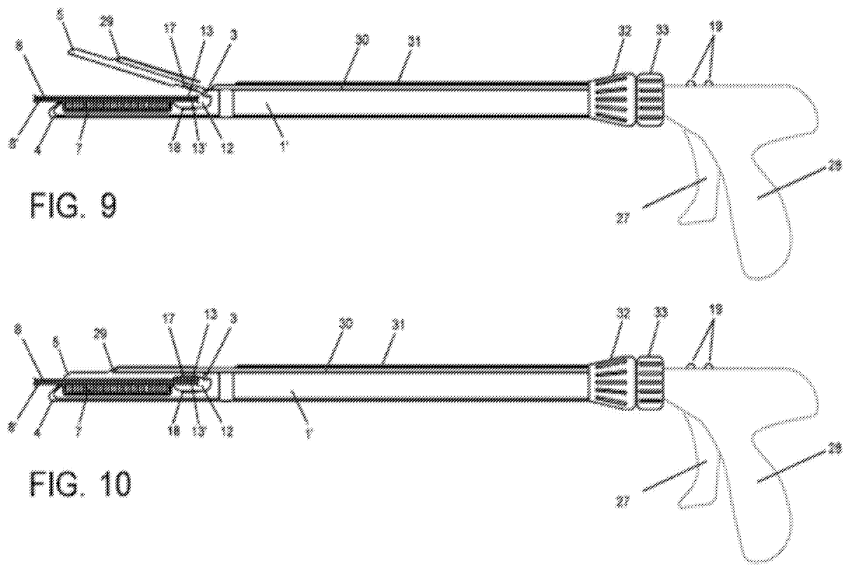

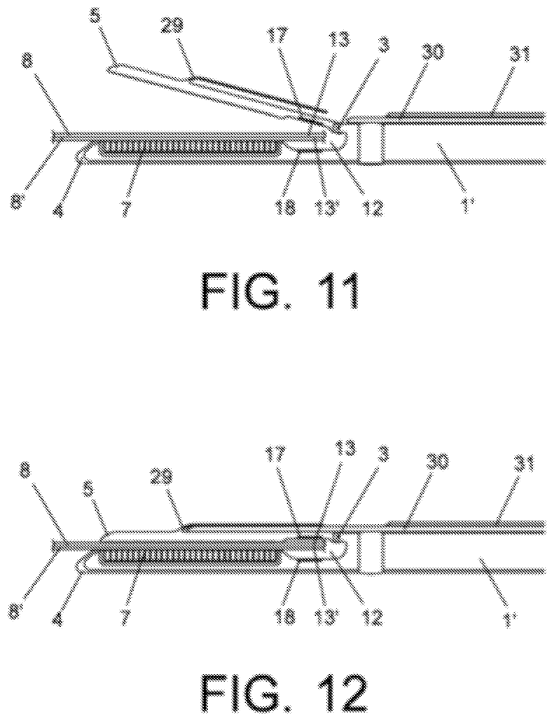

According to a variant of practical realization of the invention, the one shown in FIGS. 9 to 12, when the device is intended to be used in laparoscopic surgeries, the lower jaw (4) is subsequently prolonged in a long arm (1′) in whose bosom is axially displaced the already commented push bar that is finished on the aforementioned cam in the form of a sloping plane and which causes the sequential stapling of the tissues, all similar to as described in the first variation of realization, with the particularity that that bar is finished at its other end in a trigger thrust mechanism (27), implanted on the corresponding handle (28), which allows manual and controlled operation of that mechanism.

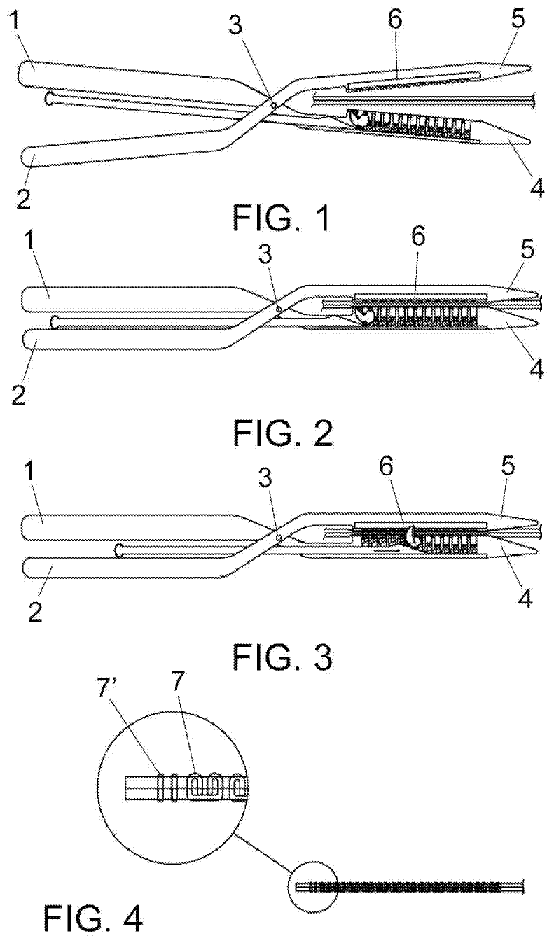

As for the means of actuating the upper jaw (5) or pressing, it shall be articulated with respect to the assembly formed by the arm (1′) and the lower jaw (4), through a axis of rotation (3), also defining a chamber (12), as well as the same stapling, cutting and sensor means described for the first example of realization, with the particularity that in order to operate that jaw it is intended that it is required to be required towards its opening position by effect of a spring associated with its axis (3) of tilting, so that, that upper jaw (5) includes in correspondence with its anterior and upper limb, a cavity (29) that is facing the upper edge of that arm (1′) laparoscopic, so that, in correspondence with that upper area of the arm, a rod (30) is axially and manually scrollable, through a guide (31) and that by virtue of such means of guidance it causes the rod to remain parallel to the laparoscopic arm (1′), to cause it, when the end of the rod (30) is inserted into the cavity (29), initially inclined, the rod (30) force the upper jaw (5) to move angularly, coinciding the position of maximum insertion of the rod (30) into the cavity (29) with the total closing position of one jaw against another. A wheel (32) geared with the rod (30), when operated, moves the rod (30) towards the cavity (29). Another wheel (33) regulates the left and right positioning of the stapler when necessary. As previously said, the other characteristics of this stapler do not vary from those described for the variation in the realization of FIGS. 1 to 6.

1. Surgical stapler, applicable to open or laparoscopic surgeries, being of the type of those constituted from a device as scissors, or tilting closure, involving two arms (1-2) of roasting and pressing, articulated on one axis (3) and topped in jaw paths (4-5), so that in one of these jaws (5) a series of conformations is established internally (6) intended to plastically deform staples (7) arranged on the complementary jaw (4), staples (7) initially set up in “U”, which are arranged on vertically scrollable grinding teeth (9) by the effect of a cam (10) in which a push bar (11) is finished horizontally within the jaw (4), where is characterised by the joint axis (3) of the two jaws (4-5) and the initial end at which the conformations are available (6) and the vertically scrollable grinding teeth (9) on which the staples are available (7), a spacing (12) is defined between the axis and the principle of the stapling of anastomosis, to preserve the part of the intestine within that space, from which the anastomosis itself is performed.

2. Surgical stapler, according to claim 1, wherein it has an IrDA type blood flow-making device, located in the cavity (12), with an emitter (17) and a receiver (18), connected to a luminous signaling device (19) located on an external arm (1) in the user's view.

3. Surgical stapler, according to claim 1, whereas it includes means of cutting to communicate the two handles to anastomose between the staple lines, materialized in a tilting blade (14) with respect to a axis of rotation (15), which is axially scrollable in a longitudinal groove of the jaw (4), against the tension of a spring, counting this tilting blade (14) with a striking roller (16) intended to come into contact with cam (10) of the thrust arm (11) during axial displacement within the jaw (4), causing its emergency and shear displacement.

4. Surgical stapler, according to claim 1, where the lower jaw (4) is subsequently extended in long arm (1′) in whose bosom the thrust bar (11) that is finished on the cam (10) in the form of a inclined plane and which causes the sequential stapling of the tissues is axially displaced, with the particularity that the rod is finished at its other end in a trigger mechanism (27), implanted on the corresponding manually operated and controlled handle of the mechanism, with the particularity that the upper jaw (5) or pressed, is articulated with respect to the assembly formed by the arm (1′) and the lower jaw (4), through a axis of rotation (3), jaw that is required towards its opening position by effect of a spring associated with its shaft (3) of tilting, so that, said upper jaw (5) includes in correspondence with its anterior and upper limb, a cavity (29) that is facing the upper edge of the aforementioned laparoscopic arm (1′), in which manually, by means of a wheel (32), geared with the rod (30), moves it through a guide (31) whose end is insertable into the cavity (29), forcing to swing to the upper jaw (5).
